(19) United States
(12) Patent Application Publication
(10) Pub. No.: US 2010/0042226 A1
(43) Pub. Date: Feb. 18, 2010

(54) ORTHOPAEDIC IMPLANT WITH SPATIALLY VARYING POROSITY

(76) Inventors: Paul S. Nebosky, Fort Wayne, IN (US); Sarah L. Zimmerman, Columbia City, IN (US); Gregory C. Stalcup, Columbia City, IN (US)

Correspondence Address: TAYLOR & AUST, P.C., P.O. Box 560, 142. S Main Street, Avilla, IN, 46710, US

(21) Appl. No.: 12/540,745

(22) Filed: Aug. 13, 2009

Related U.S. Application Data
(60) Provisional application No. 61/088,452, filed on Aug. 13, 2008.

(51) Int. Cl. A61F 2/36

(52) U.S. Cl. 623/23.3

(57) ABSTRACT

An orthopaedic implant includes a body member having a first region with a first porosity and a second region with a second porosity. At least one physical characteristic of the first porosity is different than at least one physical characteristic of the second porosity.

### CROSS REFERENCE TO RELATED APPLICATIONS

This is a non-provisional application based upon U.S. provisional patent application Ser. No. 61/088,452, entitled “ORTHOPAEDIC IMPLANT”, filed Aug. 13, 2008, which is incorporated herein by reference.

### BACKGROUND OF THE INVENTION

1. Field of the Invention

The present invention relates to orthopaedic devices, and, more particularly, to orthopaedic implants.

2. Description of the Related Art

Most orthopaedic implants are formed from a metallic material suitable for a given implant, such as a hip implant, knee implant, glenoid implant, etc. In the case of articulating joints, the implant may include a non-metallic load bearing surface, such as an ultra high molecular weight polyethylene (UHMWPE). The UHMWPE is bonded to the metallic body of the implant, and provides the implant with good wear characteristics and low friction.

It is also known to provide an implant with a porous bony ingrowth surface. For example, a hip implant may include a porous surface on the stem which is intended to allow bony ingrowth of the proximal end of the femur bone. Such a porous surface may be in the form of a metal porous surface which is bonded, such as by heat sintering, to the stem of the implant. Examples of porous surfaces of this type include a woven mesh, a fiber mesh and particles.

Porous surfaces of the type described above which are used with implants are continuous over a specified region of the implant and have the same physical properties throughout. The porous surface is formed with the intent of allowing only one type of ingrowth, such as bone ingrowth or soft tissue ingrowth.

### SUMMARY OF THE INVENTION

The present invention provides a porous ingrowth material with varying features along the length and width of an implant, such as an orthopaedic implant.

The invention in one form is directed to an orthopaedic implant, including a body member having a first region with a first porosity and a second region with a second porosity. At least one physical characteristic of the first porosity is different than at least one physical characteristic of the second porosity.

The invention in another form is directed to an orthopaedic implant, including a body member having a first porosity and a spatially discrete second porosity. At least one physical characteristic of the first porosity is different than at least one physical characteristic of the second porosity.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a stem with two different regions of ingrowth material;

FIG. 2 is a cross-section of three layers of material, the uppermost being tapered;

FIG. 3 is cross-section of multiple layers of ingrowth material with different thicknesses;

FIG. 4 is a device for genoid replacement showing porous regions optimized for: a) polyethylene molding, b) bone ingrowth, and c) soft tissue attachment and ingrowth; and

FIG. 5 is a device for tibial replacement showing porous regions optimized for: a) polyethylene molding, b) bone ingrowth, and c) soft tissue attachment and ingrowth.

Corresponding reference characters indicate corresponding parts throughout the several views. The exemplifications set out herein illustrate embodiments of the invention, and such exemplifications are not to be construed as limiting the scope of the invention in any manner.

### DETAILED DESCRIPTION OF THE INVENTION

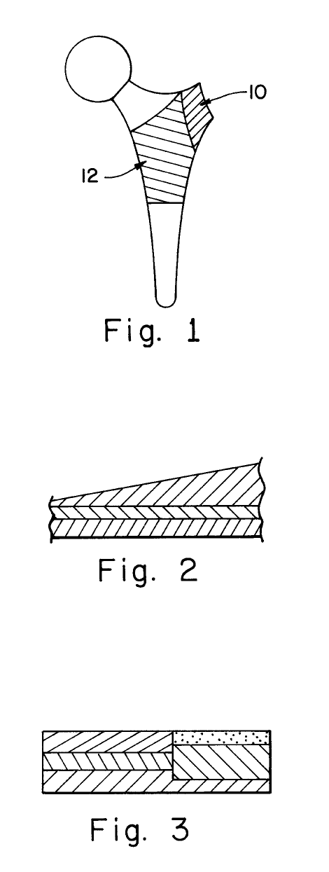

Referring now to the drawings, the ingrowth material of the present invention allows the porous material to be optimized differently for each type of tissue ingrowth desired. Examples include the following:
1. Soft tissue ingrowth in some regions and bone in another region. For example, the superior-lateral portion of a revision hip stem (FIG. 1, Ingrowth Region 10) could be optimized to encourage the ingrowth of the abductor muscle and tendon attachment, while other portions of the stem can be covered with an ingrowth material designed to encourage bone ingrowth (FIG. 1, Ingrowth Region 12). Soft tissues include, but are not limited to, tendons, ligaments, muscle, and cartilage.
2. Cortical bone ingrowth in some regions, and cancellous bone in other regions.

This type of customization of the porous ingrowth material enables an implant design that encourages ingrowth of certain tissues in some regions and occludes certain tissue ingrowth in other regions. For example, in an implant that seeks to reattach a tendon, soft tissue ingrowth may be desired in some portion of the implant, but bone ingrowth is desired in another. If one tissue or the other were to grow throughout the ingrowth material, it could prevent ingrowth of the second tissue. The ingrowth material features can be customized in each region to promote the growth of one type of tissue and/or discourage the growth of other type(s) of tissue.

The present invention further provides that therapeutic agents can be delivered to only certain regions of an implant. In implants that allow for the delivery of therapeutic agents (such as the drug delivery implants disclosed in U.S. patent application Ser. No. 12/540,676, entitled “DRUG DELIVERY IMPLANTS”, filed on even date herewith), the porous ingrowth material of the present invention can be tailored to deliver more or less of a therapeutic agent to particular tissues and regions of tissues.

This ability to tailor the implant can also aid the manufacturing process. For example, the pore shape/interconnection size can be customized to optimize insert moldability in one region and bone ingrowth in another.

A non-exhaustive list of examples of features or physical characteristics that can be varied according to the present invention include the following:
Pore size
Pore shape
Pore density (% porosity)
Pore interconnection size/shape
Surface roughness
Surface treatment (for example: a chemically etched surface in some regions and EP surface in another)
Surface coating (for example: hydroxyapatite only in some regions, an antibacterial coating in another)
Material—implantable metals, polymers, and ceramics
Ability to deliver therapeutic agents and in what amount
Specifics of the porous material (for example, in a laminate material, the thickness of one or more layers can be varied medially to laterally)
   The layer can taper (see FIG. 2)
   Layers with different, but constant, thicknesses can be stacked (See FIG. 3)
Type of Porous Material—a sintered, powdered metal ingrowth material in one region and a laminate metal in another
The scale of modifications includes macroscopic (for example pore size), microscopic (for example grain structure, microscale surface texture/treatments) and nanoscale (for example nanoscale surface texture/treatments).

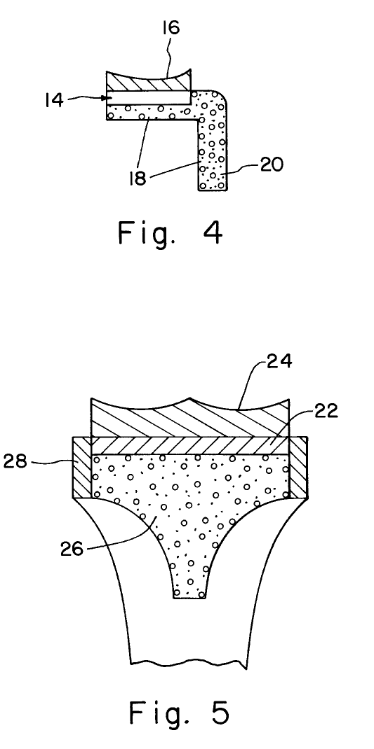

Any or all of the features described herein can be varied within the ingrowth material of a given implant. This can be applied to any implant with tissue ingrowth material (examples include, but are not limited to, primary and revision hip, knee, shoulder, and elbow prostheses; cartilage replacement plugs; soft tissue attachment screws; and spinal fusion devices). Two examples of such devices are shown in FIGS. 4 and 5. Referring to FIG. 4, a glenoid replacement is shown having a porous region 14 designed for polyethelene molding (with polyethylene 16 affixed thereto), a porous region 18 designed for bone ingrowth, and a porous region 20 designed for soft tissue ingrowth and attachment. Referring to FIG. 5, a tibial replacement is shown having a porous region 22 designed for polyethelene molding (with polyethylene 24 affixed thereto), a porous region 26 designed for bone ingrowth, and a porous region 28 designed for soft tissue ingrowth and attachment.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An orthopaedic implant, comprising:
a body member including a first region with a first porosity and a second region with a second porosity, at least one physical characteristic of said first porosity being different than at least one physical characteristic of said second porosity.

2. The orthopaedic implant of claim 1, wherein each said at least one physical characteristic includes one of: pore size; pore shape; pore density (% porosity); pore interconnection size/shape; surface roughness; surface treatment; surface coating; material type; ability to deliver therapeutic agents and in what amount; laminate thickness; taper of layer(s); thickness of layer(s); and structural type of porous material.

3. The orthopaedic implant of claim 2, wherein said structural type of porous material includes one of a sintered metal, powdered metal, and laminate metal.

4. The orthopaedic implant of claim 1, wherein each said at least one physical characteristic includes one of: a macroscopic physical property, a microscopic physical property, a microscale physical property, and a nanoscale physical property.

5. The orthopaedic implant of claim 1, wherein said first porosity and said second porosity each include at least one of a mesh, fibers, particles, beads, and a lattice.

6. The orthopaedic implant of claim 5, wherein said lattice includes a plurality of layers.

7. The orthopaedic implant of claim 6, wherein said lattice is one of a metal lattice and a ceramic lattice.

8. The orthopaedic implant of claim 1, wherein said second member is comprised of one of a metal, ceramic and a polymer.

9. The orthopaedic implant of claim 1, wherein said second member is comprised of a plurality of stacked layers which are bonded together.

10. The orthopaedic implant of claim 1, wherein said second member carries a therapeutic agent.

11. An orthopaedic implant, comprising a body member including a first porosity and a spatially discrete second porosity, at least one physical characteristic of said first porosity being different than at least one physical characteristic of said second porosity.

12. The orthopaedic implant of claim 1, wherein each said at least one physical characteristic includes one of: pore size; pore shape; pore density (% porosity); pore interconnection size/shape; surface roughness; surface treatment; surface coating; material type; ability to deliver therapeutic agents and in what amount; laminate thickness; taper of layer(s); thickness of layer(s); and structural type of porous material.

13. The orthopaedic implant of claim 12, wherein said structural type of porous material includes one of a sintered metal, powdered metal, and laminate metal.

14. The orthopaedic implant of claim 11, wherein each said at least one physical characteristic includes one of: a macroscopic physical property, a microscopic physical property, a microscale physical property, and a nanoscale physical property.

15. The orthopaedic implant of claim 11, wherein said first porosity and said second porosity each include at least one of a mesh, fibers, particles, beads, and a lattice.

16. The orthopaedic implant of claim 15, wherein said lattice includes a plurality of layers.

17. The orthopaedic implant of claim 16, wherein said lattice is one of a metal lattice and a ceramic lattice.

18. The orthopaedic implant of claim 11, wherein said first porosity and said second porosity are comprised of one of a metal, ceramic and a polymer.

19. The orthopaedic implant of claim 11, wherein said first porosity and said second porosity are comprised of a plurality of stacked layers which are bonded together.

20. The orthopaedic implant of claim 11, wherein at least one of said first porosity and said second porosity carry a therapeutic agent.
